(19) United States
(12) Patent Application Publication
(10) Pub. No.: US 2003/0220692 A1
(43) Pub. Date: Nov. 27, 2003

(54) Preparations of nucleus pulposus cells and methods for their generation, identification, and use

(76) Inventors: Irving M. Shapiro, Philadelphia, PA (US); Ramesh Rajpurohit, Hillsborough, NJ (US); Paul Ducheyne, Rosemont, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/359,419

(22) Filed: Feb. 6, 2003

Related U.S. Application Data
(60) Provisional application No. 60/354,956, filed on Feb. 9, 2002.

(51) Int. Cl. A61F 2/44

(52) U.S. Cl. 623/17.16; 435/398

(57) ABSTRACT

The present invention is directed to novel compositions and methods for the treatment of degenerative intervertebral disc disease. In some embodiments, the invention relates to a preparation of nucleus pulposus cells comprising purified nucleus pulposus cells. In some embodiments, the invention relates to methods of treating degenerative intervertebral disc disease in an individual comprising implanting nucleus pulposus cells into the nucleus pulposus space of a degenerated disc of the individual. Other embodiments of the invention relate to methods of generating nucleus pulposus cells.

### CROSS-REFERENCE TO RELATED APPLICATIONS

This claims the benefit of priority under 35 U.S.C. §119(e) from provisional U.S. Application Serial No. 60/354,956, filed on Feb. 9, 2002, which is incorporated herein by reference in its entirety.

### GOVERNMENT RIGHTS

Portions of the work related to the present inventions were funded by the National Institute of Health under Grant No. DE 13051. The United States government may therefore have certain rights to these inventions.

### FIELD OF THE INVENTION

The present invention relates to novel compositions and methods for the treatment of degenerative intervertebral disc disease involving implanting nucleus pulposus cells into the nucleus pulposus space of a degenerated disc.

### BACKGROUND OF THE INVENTION

Degenerative disease of the spine is irreversible and leads to pain, dysfunction, and loss of mechanical integrity. The Frequence of Occurrence, Impact and Cost of Musculoskeletal Conditions in the United States (Grazier, K. L. ed., 1984); Miller, J. A. A., et al., Spine, 1988, 13, 173; Boden, S. D., et al., J. Bone Joint Surg, 1990, 72A, 403; Weisel, S. A., et al., Spine, 1984, 9, 549. Environmental factors and aging contribute to disc degeneration, which is common in populations that engage in heavy physical loading, lifting, bending, twisting, and prolonged sitting and driving. Svensson, H-O, et al., Spine, 1983, 8, 272. Lumbar intervertebral disc calcification has been found in a majority of the elderly, particularly in patients suffering from osteoarthritis. Cheng, X. G., et al., Skeletal Radiology, 1996, 25, 231. Destructive lesions in cervical discs, and occasionally in lumbar discs, have been identified in rheumatoid arthritis. Milgram, J. W., Spine, 1982, 7, 498; Anonymous, International Surgery, 1968, 50, 222; Fujiwara, A., et al., European Spine Journal, 1999, 8, 396.

Proper mechanical functioning of the intervertebral disc depends to a large extent on hydration of the tissue, which decreases with age. Loss of fluid in the intervertebral disc tissue is sufficient to cause noticeable changes in disc height, which results in excessive joint load, leading to osteoarthritis. Despite the wide-spread occurrence of disc degeneration, very little work has been aimed towards understanding the biology of the cellular components that comprise the intervertebral disc and enveloping tissues.

The intervertebral disc is a critical component of the spine motion segment, which consists of an intervertebral disc sandwiched between two vertebrae, the two zygapophysial joints and capsules, and associated ligaments and muscles. The intervertebral disc is composed of three distinct tissues, namely the vertebral end-plates, annulus fibrosus (AF), and nucleus pulposus (NP), which differ widely in their matrix biology.

The vertebral end-plates are composed of hyaline cartilage and enclose the proximal and distal surfaces of the NP. The cells of the vertebral end-plates are polygonal and flattened, and are embedded in a hydrated proteoglycan gel reinforced with collagen fibrils. The morphology of the end-plate cells is similar to that of cells of the articular cartilage of synovial joints.

The AF consists of coaxial lamellae that form a helical tube that surrounds the NP. The thick collagen fibers of the AF prevent shearing of the NP and contain it during compression of the intervertebral disc.

The NP comprises the central soft portion of the disc, is mucoid in texture, and generally has a cell population of about 4000 cells/mm³, which is the lowest cell population of any connective tissue. Maroudas, The Biology of the Intervertebral Disc (Ghosh, P., ed.); The Biology of the Intervertebral Disc 1037 (Vol. 2 CRC Press 1988). About 80% of the weight of the NP constitutes water. The extracellular matrix of the NP is made up of highly hydrated proteoglycans enriched with sulfated glycosaminoglycans. Urban, J., Clin. Rheum. Dis., 1980, 6, 51. Degeneration of the NP is associated with loss of the water binding functionality of the proteoglycans, and results a progressive inability of the NP to distribute compressive loads uniformly to the surrounding AF.

Effective treatments for degenerative disc disease have yet to be developed. Existing treatments are generally limited to removing part of a disc or an entire disc, and include disectomy or spinal fusion, which fail to restore proper disc function. Spinal fusion as an intervention for degenerative disc disease is typically reserved for treatment of advanced, end-stage disease. Surgical results are varied in the near term and carry significant long-term risks. Lee, C., et al., Spine, 1991, 16(6Suppl), S253; Lehmann, T. R., et al., Spine, 1987, 12, 97. Mechanical disc replacement has not become a viable clinical option, despite the development of more than 50 different types of devices. McMillin, C. R., et al., 20^{th}Annual Meeting of the Society for Biomaterials, 1994, Abstract. A need thus exists for effective, minimally invasive treatments for degenerative disc disease that do not have significant long-term risks and that yield favorable long-term results.

### SUMMARY OF THE INVENTION

The present invention is directed, in part, to novel compositions and methods for the treatment of degenerative intervertebral disc disease. In some embodiments, the invention relates to a preparation of nucleus pulposus cells comprising purified nucleus pulposus cells. In some embodiments of the invention, the purified nucleus pulposus cells are generated by isolating nucleus pulposus cells from an intervertebral disc. In some embodiments, the purified nucleus pulposus cells are generated by culturing nucleus pulposus cells under conditions effective to maintain the phenotype of the nucleus pulposus cells. In some embodiments, the purified nucleus pulposus cells are generated by culturing precursor cells under conditions effective to cause the precursor cells to differentiate into nucleus pulposus cells.

In another embodiment, the invention relates to a method of treating degenerative intervertebral disc disease in an individual comprising implanting nucleus pulposus cells into the nucleus pulposus space of a degenerated disc of the individual.

Other embodiments of the invention relate to methods of generating nucleus pulposus cells. Some embodiments of the invention relate to methods of generating nucleus pulposus cells comprising culturing nucleus pulposus cells under conditions effective to cause the cells to maintain the phenotype of the nucleus pulposus cells. Other embodiments of the invention relate to methods of generating nucleus pulposus cells comprising culturing precursor cells under conditions effective to cause the precursor cells to differentiate into nucleus pulposus cells.

Other embodiments of the invention relate to methods of identifying nucleus pulposus cells.

These and other aspects of the invention will become more apparent from the following detailed description.

### DETAILED DESCRIPTION OF ILLUSTRATIVE EMBODIMENTS

Definitions

As employed above and throughout the disclosure, the following terms, unless otherwise indicated, shall be understood to have the following meanings.

As used herein, “culturing” is intended to refer to laboratory procedures that involve placing cells in culture medium for an appropriate amount of time to allow stasis of the cells, or to allow the cells to proliferate, differentiate and/or secrete extracellular matrix.

As used herein, “culture vessel” refers to any container in which cells may be cultured. Culture vessels include, but are not limited to, tissue culture flasks, 96 well plates, culture dishes, culture slides, and rotating wall vessels.

As used herein, “rotating wall vessel” is intended to refer to any culture vessel in which cells may be maintained in suspension during culturing. Examples of rotating wall vessels include, but are not limited to high aspect rotating vessels or rotating wall vessels fabricated by Synthecon, Houston, Tex.

As used herein, “exogenously cultured” refers to cells that have been placed in culture medium for an appropriate amount of time to allow stasis of the cells, or to allow the cells to proliferate, differentiate and/or secrete extracellular matrix.

As used herein, “preparation” refers to a collection of cells, purified such that it is substantially free from other types of cells. A cell preparation as contemplated herein is such a collection of purified cells wherein the number of cells present is useful for tissue reformation in accordance with other aspects of the invention. It is understood by those skilled in the art that limited quantities of cells for experimental or laboratory use that have been purified can be obtained by number of crude methods. Cellular preparations comprising nucleus pulposus cells in accordance with the present invention, however, are generated efficiently and in suitable quantities for use in reforming intervertebral disc tissue.

As used herein, “purified” refers to cells that are substantially free from other types of cells.

As used herein, “substantially free from other types of cells” refers to cells that are at least 80% free from other types of cells, preferably at least 90% free from other types of cells, more preferably at least 95% free from other types of cells, more preferably at least 98% free from other types of cells, more preferably at least 99% free from other types of cells, and most preferably 100% free from other types of cells.

As used herein, “precursor cells” refers to cells that, when cultured under appropriate conditions, develop into cells that possess the structure of, and function as, nucleus pulposus cells. Precursor cells include, but are not limited to, cells of the inner annulus fibrosus and nucleus pulposus.

As used herein, “nucleus pulposus cells” refers to cells that possess the structure of, and function as, nucleus pulposus cells. Nucleus pulposus cells occupy the intervertebral disc, are relatively few in number, and are surrounded by a hydrated (water containing) extracellular matrix that contains a high concentration of proteoglycan. Generally, the cells are grouped together, with about 15 to 20 cells in a group. The cells display prominent nuclei and are loaded with vesicles containing proteoglycans. Nucleus pulposus cells are present in the soft central portion of intervertebral discs and are mucoid in texture. Nucleus pulposus cells act as a cushion between the vertebrae by absorbing shock, and facilitate movement of the vertebral column.

As used herein, “phenotype of nucleus pulposus cells” is intended to refer to the presence in nucleus pulposus cells of DNA, RNA, or proteins that serve as phenotypic markers and that allow nucleus pulposus cells to be distinguished from other types of cells. Nucleus pulposus phenotypic markers include, but are not limited to, hypoxia inducing factor-1α(HIF-1 α), hypoxia inducing factor-1β (HIF-1β), glucose transporter-1 (GLUT-1), matrix metalloprotease-2 (MMP-2), lactate dehydrogenase-A (LDH-A), and thrombospondin-1 (TSP-1). “The phenotype of nucleus pulposus cells” can also refer to the morphological characteristics of nucleus pulposus cells.

As used herein, “morphological characteristics” is intended to refer to the form and structure of cells, and includes, but is not limited to, the shape and organization of cells, and the pattern formed by groups of cells.

As used herein, “differentiate” or “differentiation” is intended to refer to the development of cells with specialized structure and function from unspecialized or less specialized precursor cells, and includes the development of cells that possess the structure and function of nucleus pulposus cells from precursor cells.

As used herein, “carrier” refers to any particulate carrier, and includes, but is not limited to, microspheres and microcarrier felts. In some embodiments, carriers are preferably larger than 1 micron in diameter and less than 5 millimeters in diameter.

As used herein, “biologically active molecules” refers to those organic molecules that have an effect in a biological system, whether such system is in vitro, in vivo, or in situ. Biologically active molecules include, but are not limited to, the following: growth factors, preferably bone growth factors, cytokines, antibiotics, anti-inflammatory agents, analgesics, and other drugs. In some embodiments of the invention, biologically active molecules, include, but are not limited to, TGF-β, PDGF, EGF, FGF, IL-1, and IL-6.

As used herein, “bioactive glass” is intended to refer to any biologically active and biocompatible glass, glass-ceramic, or ceramic, including melt-derived glass and sol gel glass, which can bond to living tissue, such as bone. Bioactive glass is described in U.S. Pat. No. 5,204,106, hereby incorporated herein by reference in its entirety. Bioactive glass can be modified at its surface. Surface-modified bioactive glass is described in U.S. Pat. No. 6,224,913, hereby incorporated herein by reference in its entirety. Bioactive glass may be obtained from commercial sources such as Mo-Sci (Rolla, Mo.).

As used herein, “phenotypic marker” refers to a visible or otherwise measurable physical or biochemical characteristic.

As used herein, “implanting” is intended to refer to introducing nucleus pulposus cells with or without carriers into the nucleus pulposus space by any means effective to introduce the cells into the space.

As used herein, “individual” is intended to refer to a living mammal and includes, without limitation, humans and other primates, livestock such as cattle, pigs, horses, sheep and goats, and laboratory animals such as cats, dogs, rats, mice and guinea pigs.

As used herein, “bind” or “bound” or “bond” and all variations thereof, refers to attachment by any means, including, but not limited to, electrostatic interactions, hydrogen bonds, covalent bonds, and ionic bonds.

As used herein, “about” is intended to refer to plus or minus 10%.

As used herein, the term “sample” refers to biological material. The sample assayed by the present invention is not limited to any particular type. Samples include, as non-limiting examples, single cells, multiple cells, tissues, biological fluids, biological molecules, or supernatants or extracts of any of the foregoing. Examples include tissue removed during resection, blood, urine, lymph tissue, lymph fluid, cerebrospinal fluid, mucous, and stool samples. The sample used will vary based on the assay format, the detection method and the nature of the tissues, cells or extracts to be assayed. Methods for preparing samples are well known in the art and can be readily adapted in order to obtain a sample that is compatible with the method utilized.

As used herein, the term “detecting” means to establish, discover, or ascertain evidence of expression of phenotypic markers of nucleus pulposus cells. Methods of detecting gene expression are well known to those of skill in the art. For example, methods of detecting nucleus pulposus marker polynucleotides include, but are not limited of PCR, Northern blotting, Southern blotting, RNA protection, and DNA hybridization (including in situ hybridization). Methods of detecting nucleus pulposus marker polypeptides include, but are not limited to, Western blotting, ELISA, enzyme activity assays, slot blotting, peptide mass fingerprinting, electrophoresis, and immunohistochemistry. Other examples of detection methods include, but are not limited to, radioimmunoassay (RIA), chemiluminescence immunoassay, fluoroimmunoassay, time-resolved fluoroimmunoassay (TR-FIA), or immunochromatographic assay (ICA), all well known by those of skill in the art.

As used herein, the term “presence” refers to establishing that the item in question is detected in levels greater than background.

As used herein, the phrase “evidence of expression of nucleus pulposus phenotypic markers” refers to any measurable indicia that a nucleus pulposus phenotypic marker is expressed in the sample. Evidence of nucleus pulposus phenotypic marker expression may be gained from methods including, but not limited to, PCR, FISH, ELISA, or Western blots.

Intervertebral Disc Degeneration

Degeneration of an intervertebral disc occurs through damage to the nucleus pulposus tissue of the disc, which can be caused by aging, repetitive loading, or a significant overload. The severity of clinically observable disc degeneration varies from bulging discs to herniated or ruptured discs. Patients suffering from a degenerated disc may experience a number of symptoms, including pain of the lower back, buttocks and legs, sciatica and degenerative spondylolysis. Surprisingly, it has been discovered that nucleus pulposus cells may be implanted in the nucleus pulposus space of a degenerated disc to replace lost or damaged disc tissue, resulting in amelioration or elimination of the conditions associated with the degenerated disc. The compositions and methods of the present invention can be used to treat individuals suffering from degenerated intervertebral disc conditions, and in particular, can be used to treat humans with such conditions.

The present invention is directed to compositions and methods for the repair and/or replacement of degenerated or damaged intervertebral discs through reformation of intervertebral disc tissue. By implanting nucleus pulposus cells with or without carriers into the intervertebral space of a degenerated disc, the damaged tissue can effectively be repaired or replaced.

Some embodiments of the present invention relate to a preparation of nucleus pulposus cells comprising purified nucleus pulposus cells. In some embodiments of the invention, the purified nucleus pulposus cells are generated by isolating nucleus pulposus cells from an intervertebral disc. In some embodiments of the invention, the purified nucleus pulposus cells are generated by culturing nucleus pulposus cells under conditions effective to maintain the phenotype of the nucleus pulopsus cells, or, in other embodiments of the invention, by culturing precursor cells under conditions effective to cause the precursor cells to differentiate into nucleus pulposus cells.

Identification and Isolation of Nucleus Pulposus Cells

Some embodiments of the invention relate to a preparation of nucleus pulposus cells comprising purified nucleus pulposus cells that are generated by isolating nucleus pulposus cells from an intervertebral disc. Nucleus pulposus cells can be identified using techniques known to the art-skilled, and include recognition of the distinct morphology of nucleus pulposus cells and recognition of phenotypic markers characteristic of nucleus pulposus cells.

Nucleus pulposus cells can be identified through recognition of the distinct morphology of nucleus pulposus cells. Nucleus pulposus cells tend to form clumps of cells of about five to ten cells per clump, with what appears to be a stained material around the clump. Nucleus pulposus cells are characterized by large size, polygonal shape, and heavy vacuolation with several elongated processes, and contain considerable quantities of proteoglycans.

Nucleus pulposus cells can also be identified, prior to isolation, through recognition of phenotypic markers characteristic of nucleus pulposus cells. Phenotypic markers characteristic of nucleus pulposus cells have been ascertained by identifying gene products whose expression is upregulated in response to the conditions present in the nucleus pulposus. While nucleus pulposus cells share some of the characteristics of cartilage cells, they are embedded in a unique anatomical location that influences their biochemical and physiological characteristics. Nucleus pulposus tissue is highly avascular, and the near absence of a vascular system imposes severe restrictions on the availability of oxygen, nutrients, and growth factors to the cells. In addition, the osmotic strength of the extracellular matrix is high, while the pH is low. To survive these hostile conditions, nucleus pulposus cells have modified their biosynthetic pathways through the expression of a unique set of genes. The increased expression of certain proteins and genes in response to severe oxygen and nutrient restriction provides a molecular profile that can be used to distinguish nucleus pulposus cells from cells of the surrounding tissues.

When the oxygen concentration is low, cells rely on the glycolytic pathway to generate energy, resulting in an increased synthesis of glycolytic enzymes and an accumulation of the end products of anaerobic metabolism. Increased glycolytic activity can be mediated by HIF-1, a transcription factor that transactivates hypoxia-sensitive genes. The HIF-1α subunit is rapidly degraded under normal conditions in hypoxic tissues. HIF-1α accumulates, however, when it forms a stable heterodimer with the HIF-1β subunit. When heterodimer formation occurs, the level of HIF-1α is generally two to five times greater than that of HIF-1β. In addition, the expression of the glucose transporter protein (GLUT-1) is elevated when the expression of HIF is increased. MMP-2, a protein known to be expressed by nucleus pulposus cells, has been linked to hypoxia and disc disease. Krtolica, A. et al., Cancer Res., 1996, 56, 1168; Sedowofia, K. A. et al., Spine, 1982, 7, 213.

A variety of techniques known to those skilled in the art may be used to identify phenotypic markers of nucleus pulposus cells and differentiate nucleus pulposus cells from cells of the neighboring tissues. Such markers include, but are not limited to, expression of HIF-1α and GLUT-1, and increased expression of HIF-1β and MMP-2 relative to the levels of expression found in annulus fibrosus and end plate cells. The skilled artisan will readily appreciate that methods including, but not limited to, Western blotting, immunoprecipitation, RT-PCR, and combinations thereof, can be used to identify additional phenotypic markers for nucleus pulposus cells.

In some embodiments, the invention relates to methods of identifying nucleus pulposus cells. Such methods, in some embodiments of the invention, involve obtaining a sample to be tested for the presence of nucleus pulposus cells and detecting evidence of expression of nucleus pulposus phenotypic markers in the sample. Evidence of expression of nucleus pulposus phenotypic markers in the sample indicates the presence of nucleus pulposus cells in the sample. Nucleus pulposus phenotypic markers include, but are not limited to, HIF-1α, HIF-1β, MMP-2, MMP-9, GLUT-1, LDH-A and Thrombospondin I.

Methods for detecting evidence of expression of nucleus pulposus phenotypic markers are well known to those of ordinary skill in the art and include, but are not limited to, PCR, Northern blotting, Southern blotting, RNA protection, DNA hybridization (including in situ hybridization), Western blotting, ELISA, enzyme activity assays, slot blotting, peptide mass fingerprinting, electrophoresis, immunohistochemistry, radioimmunoassay (RIA), chemiluminescence immunoassay, fluoroimmunoassay, time-resolved fluoroimmunoassay (TR-FIA), and immunochromatographic assay (ICA).

After nucleus pulposus cells have been identified, they can be isolated from an intervertebral disc using surgical tools familiar to one of ordinary skill in the art and methods that the skilled artisan can adapt to meet the needs of the present invention.

Identification and Isolation of Precursor Cells

Some embodiments of the invention relate to a preparation of nucleus pulposus cells comprising purified nucleus pulposus cells that are generated by culturing precursor cells under conditions effective to cause the precursor cells to differentiate into nucleus pulposus cells. Nucleus pulposus precursor cells include, but are not limited to, cells of the inner annulus fibrosus.

Precursor cells can be identified using numerous methods familiar to one of ordinary skill in the art. In some embodiments of the invention, precursor cells of the inner annulus fibrosus can be identified through recognition of the distinct morphology of cells of the inner annulus fibrosus. Once identified, and then isolated, precursor cells can be cultured under conditions effective to cause the cells to differentiate into nucleus pulposus cells.

In some embodiments of the invention, precursor cells can be identified by localizing proliferative centers in the disc unit. Proliferative centers can be identified by various methods familiar to the art-skilled, including determination of the pattern of bromodeoxy-uridine (BrdU) incorporation over time into the DNA of cells of different regions of the disc, including the annulus fibrosus, vertebral end plates, and nucleus pulposus.

An actively replicating population of cells exists within the inner annulus fibrosus and outer nucleus pulposus, while cells of the inner nucleus pulposus are relatively quiescent. Although not wishing to be bound by any theory, it is thought that cells of the nucleus pulposus are generated by differentiation of cells of the inner annulus fibrosus into nucleus pulposus cells and migration of the differentiated cells into the nucleus pulposus.

In some embodiments of the invention, nucleus pulposus precursor cells can be isolated by identifying cells of the inner annulus fibrosus and isolating such cells. The distinct morphology of cells of the annulus fibrosus can be used to identify cells of the inner annulus fibrosus and to distinguish such cells from other cell types. The precursor cells can then be cultured under conditions effective to cause the cells to differentiate into nucleus pulposus cells.

The annulus fibrosus is a thick, highly organized, collagenous ligament-like structure surrounding the dorsal and lateral portions of the disc. The cells are fibroblasts and are characterized by a distinct morphology and phenotype. Microscopically, cells of the annulus fibrosus are elongated with cytoplasmic processes extending into and between the collagen bundles. The fibroblasts express type I collagen and small quantities of proteoglycans such as decorin and biglycan.

In some embodiments of the invention, nucleus pulposus precursor cells are obtained by isolating cells of the inner annulus fibrosus from one or more intervertebral discs of an individual to be treated for intervertebral disc disease. In other embodiments of the invention, nucleus pulposus precursor cells are obtained by isolating inner annulus cells from individuals other than those individuals that are to be treated for intervertebral disc disease.

In some embodiments of the invention, precursor cells can be identified and isolated from tissues other than the annulus fibrosus. Such precursor cells can be identified using means familiar to the skilled artisan, and can include, for example, pluripotent or totipotent cells such as stem cells.

Precursor cells can be isolated using surgical tools familiar to one of ordinary skill in the art and methods that the skilled artisan can adapt to meet the needs of the present invention.

Cell Culture

Some embodiments of the invention relate to methods of culturing precursor cells, such as, for example, cells of the inner annulus, under conditions effective to cause the precursor cells to differentiate into nucleus pulposus cells. Certain embodiments of the invention relate to methods of culturing self-replicating nucleus pulposus cells, such as, for example, cells of the outer nucleus pulposus, under conditions that allow the cells to proliferate and to maintain their phenotype. Some embodiments of the invention relate to preparations of nucleus pulposus cells generated by the aforementioned methods.

In some embodiments of the invention, a preparation of purified nucleus pulposus cells is generated by culturing precursor cells and/or nucleus pulposus cells in culture vessels, and preferably, in some embodiments, in rotating wall vessels, which allows the oxygen concentration and the composition of the culture medium to be modulated with high precision.

In some embodiments of the invention, a preparation of purified nucleus pulposus cells is generated by culturing precursor cells and/or nucleus pulposus cells that have been seeded onto a carrier. Accordingly, in some embodiments of the invention, the nucleus pulposus cells or precursor cells are combined with a carrier prior to, or simultaneous with, culturing. In other embodiments of the invention, the nucleus pulposus cells are combined with a carrier following culturing. In some embodiments of the invention, a preparation of purified nucleus pulposus cells is generated by culturing precursor cells and/or nucleus pulposus cells in culture vessels, and preferably, in petri dishes, in the absence of carrier materials.

In some embodiments of the invention, a surface-modified (i.e., containing a calcium phosphate surface film) bioactive glass carrier is used as a substrate for nucleus pulposus cell attachment and proliferation. In some embodiments of the invention, the carrier is a composite bioactive, biodegradable microsphere, as described in U.S. Pat. No. 6,328,990, hereby incorporated by reference in its entirety. In some embodiments of the invention, the carrier can be fabricated as described in U.S. Pat. No. 6,328,990 using a solid-in-oil-in-water (s/o/w) emulsion solvent removal method to incorporate modified bioactive glass powders (MBG) into a degradable polylactic acid (PLA) polymer matrix to form composite microspheres. In some embodiments of the invention, the carrier accumulates a bioactive calcium phosphate surface film after immersion in simulated physiological solution.

In accordance with some embodiments of the present invention, the carrier is comprised of bioactive glass. Bioactive glass is described in Ducheyne, P., J. Biomedical Materials Res., 1985, 19, 273; Brink, M., et al., J. Biomed Master Res., 1997, 37, 114, and U.S. Pat. No. 5,204,106, hereby incorporated by reference herein in their entireties. A typical bioactive glass composition contains oxides of silicon, sodium, calcium and phosphorous in the following percentages by weight: about 40% to about 60% SiO₂, about 10% to about 30% Na₂O, about 10% to about 30% CaO, and 0% to about 10% P₂O₅. Other oxides can also be present in bioactive glass compositions as described in Ducheyne, P., J. Biomedical Materials Res., 1985, 19, 273 and Brink, M., et al., J. Biomed Materials Res., 1997, 37, 114. In some preferred embodiments of the invention, the nominal composition of the bioactive glass by weight is 45% SiO₂, 24.5% Na₂O, 24.5% CaO and 6% P₂O₅, and is known as 45S5 bioactive glass. Bioactive glass may be obtained from commercial sources such as Mo Sci., Inc. (Rolla, Mo.). In some embodiments the bioactive glass is sol gel.

The granule size of the bioactive glass may be selected based upon the degree of vascularity of the affected tissue. In some embodiments of the invention, the granule size will be less than about 1000 μm in diameter. In some embodiments of the present invention, it is preferred that the bioactive glass granules be from about 200 μm to about 300 μm in diameter. In some embodiments of the present invention, granule size is from about 50 μm to about 150 μm.

In some embodiments of the present invention, the bioactive glass has pores. In some embodiments of the present invention, the pore size of the bioactive glass is less than about 850 μm in diameter, while a pore diameter of about 150 μm to about 600 μm is preferred.

In some embodiments of the invention, the carrier is a porous structure, such as the porous, bioactive glass described in U.S. Pat. Nos. 5,676,720 and 6,328,990, hereby incorporated by reference in their entireties. In some embodiments of the invention the carrier is a porous felt, such as the porous metal fiber mesh described in U.S. Pat. No. 4,693,721, hereby incorporated by reference in its entirety.

In some embodiments of the invention, the apparent density of the carrier is about that of the culture medium, and is from about 0.90 g/cc³ to about 1.10 g/cc³. In some preferred embodiments of the invention, the apparent density of the carrier is slightly less than that of the culture medium, and is from about 0.95 g/cc³ to about 1.0 g/cc³.

In some embodiments of the invention, the precursor cells or nucleus pulposus cells are seeded onto carrier materials and are cultured in rotating wall vessels as described in Radin, S., et al., Biotechnology and Bioengineering, 2001, 75(3), 369 and Gao, H., et al., Biotechnology and Bioengineering, 2001, 75(3), 379. The rotating wall vessel is a microcarrier culture system in a fluid-filled vessel that rotates about a horizontal axis. The cells and carrier materials are maintained in suspension in the rotating wall vessels. Gravity-induced sedimentation is balanced with fluid drag and rotation-induced centrifugation. In a preferred embodiment, the rotating wall vessels are high aspect ratio vessels. Id.

In some embodiments of the invention, the nucleus pulposus and/or precursor cells are attached to the carrier material. In some embodiments of the invention, the cells attach to the carrier through the interaction of fibronectin with integrin receptors located on the nucleus pulposus and precursor cell surfaces. Fibronectin is selectively adsorbed by the calcium phosphate layer that forms on the bioactive glass carrier. Fibronectin binds to hyaluronic acid, which in turn binds the CD44 receptors present on the surfaces of nucleus pulposus cells and precursor cells, thus serving to attach the cells to the surface-modified bioactive glass.

The following methods can be used, in some embodiments of the invention, to isolate and culture the precursor and/or nucleus pulposus cells. Nucleus pulposus and/or annulus fibrosus tissue is removed from intervertebral discs using methods known to those skilled in the art. The tissues are treated with collagenase at about 37° C. at a concentration of about 0.1 unit/ml to about 10 unit/ml, and more preferably at about 1 unit/ml, for about 15 minutes to about 2 hours. Following collagenase treatment, the cells are swollen and easily ruptured, and are gently pipetted up and down to break up the aggregates. The cell suspensions are centrifuged at about 2500 rpm for about 5 min. The supernatant is discarded and the cell pellet is suspended in complete Dulbecco's Eagle's Medium supplemented with about 1% to about 70% fetal calf serum, and more preferably about 10% fetal calf serum, about 0.1 mM to about 20 mM, and more preferably about 2 mM, glutamine and penicillin/streptomycin/fungicide. The cells are treated with hylauronidase (about 0.1 unit/ml to about 10 unit/ml, and more preferably about 1 unit/ml) to facilitate cell attachment and are washed with complete medium, that is, medium containing 10% serum, to remove the hylauronidase.

In some embodiments of the invention, nucleus pulposus and/or precursor cells are selected after hyaluronidase treatment, thereby separating them from non-nucleus pulposus or and/or non-precursor cells, using methods familiar to the skilled artisan, such as, for example, FACS. In some embodiments of the invention, non-nucleus pulposus or non-precursor cells are removed after hylauronidase treatment using methods familiar to the skilled artisan, such as, for example, elutration, which involves differential centrifugation based upon the buoyant density of the cells, or centrifugation over a Percoll gradient.

In another embodiment of the invention, the precursor and/or nucleus pulposus cells are isolated by gently teasing out fragments of nucleus pulposus tissue from intervertebral discs. The tissue is placed in culture vessels with tissue culture medium and cells are allowed to grow out from the nucleus pulposus tissue. In 7 to 14 days the cells are released from the tissue culture plastic and collected by centrifugation. In some embodiments of the invention, nucleus pulposus and/or precursor cells are selected after collection by centrifugation according to the methods described above.

The precursor cells and/or nucleus pulposus cells, isolated by either of the methods described above, or by other methods familiar to one of ordinary skill in the art, at about 1×10⁴ cells/ml to about 1×10⁸ cells/ml, preferably at about 1×10⁵ cells/ml to about 1×10⁷ cells/ml, and more preferably at about 1×10⁶ cells/ml, and carrier are injected into culture vessels, and, preferably, rotating wall vessels, at a ratio of cells to individual carriers of about 1000:1 to about 10:1, and more preferably at about 100:1. The culture vessels are rotated at a speed of about 5 to about 20 rpm. The oxygen concentration of the medium is maintained at about 0.02% to about 20%, and more preferably at about 0.2% to about 2%. The ionic strength of the medium is adjusted using NaCl and is maintained at about 100 mOsmols to about 900 mOsmols, and more preferably at about 280 mOsmols to about 450 mOsmols. The pH of the medium is maintained at about 6.5 to about 7.9 by the addition of 10 mM HEPES. The glucose concentration in the medium is maintained at about 2 to about 10 g/L. The temperature of the medium is maintained at about 35 to about 40° C.

In some embodiments of the invention, the medium is supplemented with fibronectin at about 0.0001 to about 1 mg/ml. In some embodiments of the invention, the medium is supplemented with TGF-β at about 10 picograms/ml to about 10,000 picograms/ml, and more preferably at about 100 picograms/ml to about 1000 picograms/ml; with PDGF at about 1.0 ng/ml to about 10,000 ng/ml, and more preferably at about 10 ng/ml to about 1000 ng/ml; with EGF at about 0.5 ng/ml to about 150 ng/ml, and more preferably at about 1.0 ng/ml to about 10 ng/ml; with FGF at about 0.5 ng/ml to about 150 ng/ml, and more preferably at about 1.0 ng/ml to about 10 ng/ml; with IL-1 at about 0.5 ng/ml to about 150 ng/ml, and more preferably at about 1.0 ng/ml to about 10 ng/ml; and with IL-6 at about 0.5 ng/ml to about 150 ng/ml, and more preferably at about 1.0 ng/ml to about 10 ng/ml. The medium is replenished every two days. The growth and development of the cells are monitored by the removal of an aliquot of microcarrier from the culture about every two days and determining the DNA content of the cells.

In some embodiments of the invention, the precursor cells, or the nucleus pulposus cells, and carrier, are combined with biologically active molecules. In some embodiments of the invention, the precursor cells, or nucleus pulposus cells, and carrier, are combined with at least one biologically active molecule prior to injection of the cells and carrier into the culture vessels. In some embodiments of the invention, the biologically active molecules are contained within or upon the carrier. In some preferred embodiments of the invention, the biologically active molecules contained within the carrier are released from the carrier in a controlled release manner during culture and/or after implantation into the nucleus pulposus space, as described in U.S. Pat. No. 5,591,453, hereby incorporated by reference in its entirety. In some embodiments, the biologically active molecules comprise growth factors, cytokines, antibiotics, proteins, anti-inflammatory agents, or analgesics. Preferred biologically active molecules include TFG-β, PDGF, EGF, FGF, IL-1 and IL-6.

In some embodiments of the invention, maintenance of the phenotype of the nucleus pulposus cells during culture of nucleus pulposus cells, and differentiation of precursor cells into nucleus pulposus cells during culture of precursor cells, are determined using means familiar to the skilled artisan, which include, but are not limited to, biological assay of the cells for the expression of phenotypic markers of nucleus pulposus cells using Western blotting, immunoprecipitation, and RT-PCR techniques.

In some embodiments of the invention, maintenance of the phenotype of the nucleus pulposus cells during culture of nucleus pulposus cells, and differentiation of precursor cells into nucleus pulposus cells during culture of precursor cells, are determined by examination of the morphology of the cultured cells. The morphology of the cells may be examined by means familiar to the skilled artisan, which include, but are not limited to, viewing with the naked eye or viewing under a light or electron microscope. Nucleus pulposus cells have a characteristic morphology that includes the formation of clumps of cells of about five to ten cells per clump, with what appears to be a stained material around the clump. The cells are highly vacuolated and contain considerable quantities of proteoglycans.

Methods of Treatment

Treatment of Initial Stages of Intervertebral Disc Disease

Some embodiments of the invention include methods of treating the initial stages of degenerative intervertebral disc disease in an individual, and involve minimally invasive surgical techniques, such as the implantation of a biomaterial scaffold and/or nucleus pulposus cells into the nucleus pulposus space of the individual. Biomaterial scaffolds are described in U.S. Pat. No. 5,964,807, incorporated herein by reference in its entirety.

Some embodiments of the invention involve implanting a biomaterial scaffold directly into the nucleus pulposus space with one or more percutanous injections. In some embodiments of the invention, the biomaterial scaffold comprises biologically active glass, as previously described. In some embodiments of the invention, the scaffold further comprises biologically active molecules. In some embodiments of the invention, the scaffold is combined with one or more pharmaceutically acceptable excipients prior to implantation into the nucleus pulposus space. Pharmaceutically acceptable excipients are familiar to the skilled artisan and include, but are not limited to, buffers, physiological saline, and viscous fluids that harden into a gelatinous composite, such as, for example, self-setting hydrogel and alginate. Implantation of the biomaterial scaffold into the nucleus pulposus space leads to regeneration of nucleus pulposus cells with concomitant restoration of the function of the nucleus pulposus tissue.

Some embodiments of the invention involve implanting nucleus pulposus cells into the nucleus pulposus space of a degenerated disc of an individual by making one or more percutanous injections with a needle. Ultrasound or other imaging techniques can be used to guide the needle to the nucleus pulposus space. In some embodiments of the invention, after implantation into the nucleus pulposus space, the nucleus pulposus cells continue to proliferate and expand, thereby regenerating nucleus pulposus tissue and reestablishing the natural function of the degenerated disc.

In some embodiments of the invention, the nucleus pulposus cells are combined with one or more pharmaceutically acceptable excipients, as described above, prior to implantation into the nucleus pulposus space. In some embodiments of the invention, the nucleus pulposus cells are combined with biologically active molecules prior to implantation into the nucleus pulposus space.

In some embodiments of the invention, nucleus pulposus cells are generated by culturing nucleus pulposus cells and/or precursor cells, and the cells are then implanted into the nucleus pulposus space of a degenerated disc of an individual to be treated. In some embodiments of the invention, following cell culture, and prior to implantation into the nucleus pulposus space, contaminating non-nucleus pulposus cells are removed from the exogenously-cultured nucleus pulposus cells using methods familiar to one of ordinary skill in the art. In some embodiments of the invention, the exogenously cultured nucleus pulposus cells are removed from the carrier material upon which they were seeded during culture prior to implantation of the cells into the nucleus pulposus space.

Treatment of Advanced Stages of Intervertebral Disc Disease

Some embodiments of the invention involve methods of treating the advanced stages of intervertebral disc disease in an individual. Some embodiments of the invention involve implanting nucleus pulposus cells into the nucleus pulposus space as part of a larger substrate, which includes, in some embodiments of the invention, carrier material upon which the cells were seeded during culture.

In accordance with some embodiments of the present invention, the carrier is biodegradable, which means that, after implantation of nucleus pulposus cells into a degenerated disc, the carrier degrades into natural, biocompatible byproducts over time until the carrier is substantially eliminated from the implantation site and, ultimately, the body. In accordance with some embodiments of the present invention, the rate of biodegradation of the carrier is less than or equal to the rate of intervertebral disc tissue formation such that the rate of tissue formation is sufficient to replace the carrier that has biodegraded.

In some aspects of the present invention, the biodegradable carrier is bioactive, which means that the carrier enhances cell function. For instance, bioactive glass granules have been shown to enhance cell growth of typical bone cells. Schepers et al., U.S. Pat. No. 5,204,106. In addition, dense bioactive glass discs have been found to enhance osteoprogenitor cell differentiation beyond the levels of enhanced differentiation elicited by bone morphogenic protein. H. Baldick, et al., Transactions 5th World Biomaterials Conference, Toronto, II-114 (June, 1996).

In some embodiments of the invention, the biodegradable carrier has sufficient mechanical strength to act as a load bearing spacer until intervertebral disc tissue is reformed. In some embodiments, the biodegradable carrier is biocompatible such that it does not elicit an immune or inflammatory response that might result in rejection of the implanted material.

In some embodiments of the invention, the nucleus pulposus space of the degenerated disc to be treated by the methods of the invention is evacuated prior to implantation of the nucleus pulposus cells. In other embodiments of the invention, the nucleus pulposus space is evacuated after implantation of the nucleus pulposus cells. Preferably, for treatment of advanced stages of intervertebral disc disease, the nucleus pulposus space of the degenerated disc is evacuated prior to implantation of the nucleus pulposus cells.

Evacuation of the degenerated intervertebral disc tissue, and primarily the nucleus pulposus tissue, is performed using known surgical tools with procedures adapted to meet the needs of the present invention. For example, an incision or bore may be made at the lateral edge in the annulus fibrosus and the intervertebral disc tissue is extracted from the nucleus pulposus via, for example, the guillotine cutting approach. The tissue can be extracted using a scalpel, bore, or curette. Alternatively, the tissue may be aspirated. In some embodiments, the annulus fibrosus, or significant portions thereof, is left intact. It is preferred in some embodiments of the invention that at least 50% of the annulus fibrosus remains intact. It is more preferred in some embodiments that at least 85% of the annulus fibrosus remains intact. Arthroscopic techniques are most preferred in accordance with methods of the present invention.

Where delay occurs between evacuation of nucleus pulposus tissue and implantation of the exogenously cultured nucleus pulposus cells, the evacuated space may be temporarily filled with gel foam or other load bearing spacers known in the art.

In some embodiments of the invention, the previously described methods for treating intervertebral disc disease are used in conjunction with other known, conventional treatments.

The methods of the present invention provide advantages over methods of the prior art because an entire degenerated disc does not need to be removed for treatment of the disc. Rather, in some embodiments of the invention, only the nucleus pulposus space of a degenerated disc is evacuated. The present invention thus, in some embodiments, provides less invasive procedures than those of the prior art. In addition, the compositions and methods of the present invention prompt biological repair of normal tissue in the disc, which results in better long term results than those obtained with synthetic prostheses.

The materials, methods and examples presented herein are intended to be illustrative, and are not intended to limit the scope of the invention. All publications, patent applications, patents, and other references mentioned herein are incorporated by reference in their entirety. Unless otherwise defined, all technical and scientific terms are intended to have their art-recognized meanings.

### EXAMPLES

### Example 1

### Identification of Proliferative Centers in the Intervertebral Disc

Bromodeoxy-Uridine (BrdU), a thymidine analogue (10 mg/Kg bodyweight) was injected intraperitoneally into five day old mice. The animals were sacrificed at 0.5, 8 and 24 hours after BrdU injection. Incorporation of bromodeoxyuridine into the DNA of dividing cells was detected by immunohistochemical staining methods. The presence of dividing cells was confirmed by localization of the cell cycle dependant polymerase-delta accessory protein, proliferation cell nuclear antigen, by immunohistochemistry.

Freshly isolated intervertebral discs were immediately fixed in 4% neutral buffered formalin. After several changes of the formalin over a few days, and following a series of dehydration steps by graded levels of alcohol and xylene, the discs were embedded in paraffin. Transverse and coronal sections of 8-10 microns were cut and dried overnight. Tissue sections were dewaxed in xylene and taken to water through graded levels of alcohols. Endogenous peroxidase was quenched by incubation in 2% H₂O₂ in methanol at room temperature for 20 min. The tissue sections were mildly treated with trypsin. The incorporation of bromodeoxyuridine (BrdU) into DNA was detected by a monoclonal antiBrdU antibody (BrdUr Staining Kit, Oncogene Research Products, Cambridge Mass.) according to the manufacturer's protocol, and visualized using a biotin-streptavidin-peroxidase and diaminobenzidine staining system.

Examination of transverse and coronal sections of intervertebral disc cells of a 5 day old mouse injected with bromodeoxy-uridine revealed that BrdU was incorporated into the cells of the intervertebral disc in a time dependant manner. Very little BrdU was incorporated 30 minutes after injection, but incorporation increased significantly by 24 hours. Both the transverse and the coronal sections exhibited a similar pattern of staining of the BrdU positive cells. The BrdU labeling of the cells was much more intense and concentrated at the interface of the nucleus pulposus and the inner annulus. Furthermore, the incorporation of BrdU into the cells occurred largely at the two lateral ends of the disc. In the coronal sections, appreciable amounts of incorporation were seen in the proliferating region of the end plate cartilage. Very little incorporation was visible in the central core of the nucleus pulposus. A magnified view of a portion of the interevertebral disc in transverse section revealed that cells from the inner annulus migrate towards the center of the nucleus, indicating that cells of the inner annulus differentiate into nucleus pulposus cells.

Proliferating cell nuclear antigen (PCNA) was detected by using the mouse monoclonal antibody (Oncogene Research Products, Cambridge Mass.). Tissue sections were treated with hyaluronidase (1 unit/ml, Sigma Co. St. Louis Mo.) to digest the proteoglycans. The samples were then blocked by anti-mouse antibody and bovine serum albumin (1%) and incubated with the primary PCNA antibody (10 μg/ml). Staining was visualized by the peroxidase and diaminobenzidine system. Sections were counter-stained by Alcian blue. Staining of PCNA in sections of the intervertebral disc was strongly associated with most of the cells in the annulus. A few cells in the nucleus pulposus, which were located mostly at the periphery of the nucleus pulposus close to the inner annulus layer of cells, were also PCNA positive.

### Example 2

### Characterization of the Morphology of Cells of the Intervertebral Disc

Cells were isolated from different regions of the intervertebral disc from adult rats and were grown in culture for 3 to 4 weeks to characterize their morphology and proliferation rates.

Adult rats approximately 8-10 weeks of age weighing between 180-200 g were used. The animals were sacrificed and intervertebral discs from the cervical to the lumbar region of the spine were immediately obtained under aseptic conditions. Adherent ligamentous tissue was removed from the annulus, vertebral bone fragments, and the cartilage end plates of the complete intervertebral discs.

The discs were immersed in calcium and magnesium free Hanks' buffered salt solution (HBSS) supplemented with 80 mM NaCl. A cut was made through the middle of the annulus with a thin #15 scalpel blade and the two halves of the disc were held wide open to facilitate release of the contents of the disc into the high osmolality medium. To isolate cells from the annulus and the cartilage end plates, the discs were transferred to a second dish containing HBSS. Small pieces of annulus tissue from the inner one-third of the annulus, designated as the inner annulus, and the outer one-third of the annulus, designated as the outer annulus, were cut and removed. The central portion of the translucent plate of the cartilage end plate was isolated. Cells of the nucleus pulposus were treated with collagenase at 1 unit/ml for 15 min at 37° C., while cells of the inner annulus, outer annulus and end plates were treated with collagenase for 2 hours after chopping the fragments into very small pieces.

Following collagenase treatment, the nucleus pulposus cells were gently pipetted up and down to break up the cell aggregates because the cells were swollen and easily ruptured. The cells of the inner annulus, outer annulus and end plates were thoroughly agitated following collagenase treatment to break up the cell aggregates. The cell suspensions were centrifuged at 2500 rpm for 5 min. The supernatant was discarded and the cell pellet was suspended in complete Dulbecco's Eagle's Medium supplemented with 10% fetal calf serum, 2 mM glutamine and penicillin/streptomycin/fungicide and plated in 60 mm dishes. The cells in the dishes were treated with hylauronidase (1 unit/ml) to facilitate cell attachment. The medium was changed every third day. To monitor growth of the cells, the cells were counted on a hemocytometer.

After the cells had been in culture for one week, very few cells attached to the surface of the plastic dish. Treatment with hyaluronidase did not improve cell attachment. The morphology of the cells that were attached was characterized by large size, polygonal shape, and heavy vacuolation with several elongated processes. The nucleus pulposus cells grown in culture continued to maintain this morphology for up to at least 2 to 3 weeks. Cells from the different regions of the intervertebral disc had a distinct morphology. The inner and outer annulus cells appeared fibroblastic and proliferated at a very rapid rate, growing to confluency within 2 weeks. Occasionally it was possible to see a few cells bearing a strong resemblance to the morphology of the nucleus pulposus cells, in the midst of the fibroblastic inner annulus layer of cells. The end plate cells showed the characteristic chondrocytic morphology, polygonal shape and granular cytoplasm, and a small size as compared to the nucleus pulposus cells.

The relative rates of proliferation of the nucleus pulposus, inner annulus, outer annulus and end plate cells were determined. The cells of the inner and outer annulus proliferated at the fastest rate and grew to confluency within about two weeks. The end plate cells had a slower rate of growth than either the inner or the outer annulus cells, but the growth rate was at least two-fold faster than that of the nucleus pulposus cells. Culturing the nucleus pulposus cells beyond three weeks did not increase the cell number and many of the cells began to disintegrate and die, while some dedifferentiation of the nucleus pulposus cells was observed.

### Example 3

### Preparation of Hollow, Biodegradable Composite Microspheres

Six-hundred mg of polylactic acid was dissolved in 5 ml of methylene chloride and 600 mg of modified bioactive glass powder was mixed with the PLA solution via sonication for 15 min. The PLA-MBG mixture was added drop by drop to 200 ml of 0.5% (w/v) PVA solution. The mixture was vigorously stirred in a 500-ml beaker for 4 hours at room temperature. The microspheres were collected by centrifugation, filtered, washed, dried and stored in a dessicator. Subsequently, the microspheres were immersed in simulated physiological fluid for 2 weeks to form a bone bioactive apatite-like layer on their surfaces.

The morphology and chemical composition of the surface of the microcarriers were examined using scanning electron microscopy (SEM) and energy dispersive x-ray analysis (EDXA). Fourier transform infrared spectroscopy (FTIR) was performed on powder-KBr mixtures in the diffuse reflectance mode.

SEM analysis revealed that, upon the s/o/w synthesis, the composite microspheres were mostly covered by PLA, and micron-size pores existed on the microsphere surfaces. Examination of microsphere cross-sections revealed that the microspheres had a porous structure due to the solvent removal process. Modified glass powders were distributed throughout the porous polymer matrix. EDXA analysis on microsphere cross-sections further confirmed the presence of typical elements of glass, i.e. Si, Ca and P.

After 2 weeks of immersion in simulated physiological solution, the surfaces of the composite microspheres were mostly covered by small precipitates with a diameter of up to 3 μm. The precipitates consisted of assemblies of small flake-like pieces. FTIR spectra of composite microspheres immersed in simulated physiological fluid (SPF) for 1, 2 and 3 weeks revealed (PO₄)³⁻ bands at 1098, 1046, 950, 606 and 561 cm⁻¹ which can be assigned to calcium hydroxyapatite. The intensity of the P-O bands increased with incubation time.

### Example 4

### Determination of the Trajectories of the Microcarriers

The trajectories of a large number of hollow biodegradable bioactive glass-polymer composite microcarriers were determined in high aspect rotating vessels (HARV) in an inertial and a rotating frame of reference, respectively. With the progress of time, the hollow, biodegradable, composite microcarriers (ρₚ<<ρₗ) did not collide with the walls of the vessels and thus were not damaged. The microcarriers remained in the central region of the vessels, which allowed them to obtain adequate nutrition. Since the microcarriers had a low apparent specific weight slightly less than that of the medium and were hollow, they experienced very low shear stress (0.3 dynes/cm²).

### Example 5

### Surface-Modified Bioactive Glass Promotes Nucleus Pulposus Cell Proliferation

Nucleus pulposus cells were isolated from adult rabbit discs and seeded onto surface modified bioactive glass. At selected time intervals, the cells and scaffold were evaluated. The cells rapidly attached to the substrate, colonizing it within 12 hours. By 21 days a lawn of cells had formed over the substrate. DNA measurements revealed the unique phenomenon of a progressive increase in cell number with time, which was contrary to the commonly accepted view that nucleus pulposus cells have minimal proliferative activity. The phenotype of the nucleus pulposus cells was maintained as evidenced by the expression of aggrecan and collagen type II and I, and the absence of expression of collagen type X. CD44, a cell-surface glycoprotein that binds hyaluronate, was also expressed by the cells. EDXA and FTIR revealed the formation of a calcium phosphate-rich layer on the substrate surface.

### Example 6

### Identification of Phenotypic Markers for Nucleus Pulposus Cells Isolation of Nucleus Pulposus and Surrounding Tissues

Adult rats approximately 8-10 weeks of age weighing between 180-200 g were sacrificed and the spines were isolated. Ribs and other adherent structures were removed with rongeurs. Disc units (the intervertebral disc and adjacent vertebrae) from the mid-thoracic to the lumbar region of the spine were obtained under aseptic conditions. Adherent ligamentous tissue from the annulus and the vertebral bone fragments of the cartilage end plates were removed from the complete intervertebral discs. Disc units that were to be used for immunohistochemistry were fixed in 4% formalin in phosphate-buffered-saline (PBS) for 3-4 days.

Disc Cell Collection

The disc units were immersed in calcium and magnesium-free Hanks' buffered salt solution (HBSS), pH 7.4, supplemented with 80 mM NaCl. Transverse cuts parallel to the disc axes were made through the superior surface of the annulus tissues with a scalpel blade (#15), and the two halves of the discs were held open with fine forceps, which facilitated release of the contents of the discs into the high osmolality medium. The extract contained both the nucleus pulposus and the transitional zone. The transitional zone is a cell layer that abuts into the nucleus pulposus from the annulus fibrosus. The cells of the transitional zone are proliferative in nature and their morphology resembles that of cells that are seen in the nucleus pulposus.

The cells were centrifuged at 2500 rpm for 10 min. and the supernatant was removed and the cell pellet collected. The discs were then transferred to a second dish containing HBSS to isolate the annulus and the cartilage end plates. Adherent annulus tissue and cartilage end plates were then removed, resulting in isolation of only about two-thirds of the middle portion of the annulus. Small pieces of tissue from the central translucent region of the end plates were harvested. The end plate, annulus, and nucleus pulposus tissue fragments were suspended in 0.1% Triton-X 100 in PBS (v/v) containing phenyl methyl sulfonyl fluoride (0.5 μM), leupeptin (1 μg/ml), pepstatin (1 μg/ml) and aprotinin (1 μg/ml). The extracts were polytron homogenized and stored at −80° C. until they were analyzed.

Western Blotting

Extracts of nucleus pulposus, annulus and cartilage end plate cells were isolated from the disc units as described above. Equal amounts of protein were electrophoresed on SDS polyacrylamide gels (6% for aggrecan, 10% for GLUT-1, and 12% for HIF-1 subunits and MMP-2). For aggrecan, samples were incubated with 0.1 unit of chondroitinase ABC (Sigma Chemical Co., St. Louis, Mo.), in 50 mM Tris acetate, 10 mM EDTA, pH 7.6, for 1 h at 37° C. Following electrophoresis, the protein bands were transferred to a nitrocellulose membrane and treated with primary antibodies to aggrecan (1:2500), HIF-1α (1:100), HIF-1β (1:200) (Novus Biologicals, Littleton, Colo.), MMP-2 (1:200) (Chemicon Internationals Inc., Temecula, Calif.), and GLUT1 (1:200) (Santa Cruz Biotechnology Inc., Santa Cruz, Calif.). The blots were incubated with the peroxidase-labeled secondary antibody, and the protein bands were detected using the light emitting ECL™ Western blotting detection system (Amersham Pharmacia Biotech, Piscatway N.J.). Protein was measured using the DC protein assay (BIORAD Laboratories, Hercules, Calif.) according to the manufacturer's protocol.

Western blot analyses were performed with protein extracted from nucleus pulposus, annulus and end plate cartilage cells. The extracts were first examined for the presence of aggrecan and a band of about 230 kd was observed in the nucleus pulposus extracts. In contrast, very low levels of aggrecan were observed in extracts of annulus and end plate cells.

A band corresponding to HIF-1α (about 110 kd) was present in nucleus pulposus cell extracts, while neither annulus fibrosus nor end plate cells expressed the HIF-1α isoform. All three tissues expressed HIF-1β and the protein was present in significant quantities in nucleus pulposus extracts. A small band, possibly corresponding to pre-HIF-1β was evident together with some low molecular weight fragments (75-95 kd).

Expression of the glucose transporter, GLUT-1, was also examined, and only cells of the nucleus pulposus expressed the 37 kd protein. Cells of the annulus and end plates expressed very low levels of the transporter. MMP-2 expression was also examined, and high levels of the protein were observed in extracts of nucleus pulposus cells, while low levels of the enzyme were observed in the annulus and end plate extracts.

Immunohistochemistry

Following tissue fixation, disc units were embedded in paraffin and transverse and coronal sections 8-10 μm thick were prepared. The sections were deparaffinized in xylene and rehydrated through graded ethanols.

For aggrecan, samples were incubated with the primary antibody in 1% bovine serum albumin in PBS at a dilution of 1:100 at 4° C. overnight. The antibody was raised in rabbit against the peptide sequence from residues 24-151 of the mouse aggrecan core protein precursor. After thoroughly washing the sections, the bound primary antibody was incubated with horse radish-peroxidase conjugated anti-rabbit goat secondary antibody, at a dilution of 1:500 (Boehringer Mannheim Indianapolis Ind.) for 1 h at room temperature. For HIF-1 and MMP-2, the sections were first treated with 1 unit of hyaluronidase for 1 h at 37° C. The sections were washed with PBS and then were incubated with primary antibodies of HIF-1α (1:10) and β (1:20) (Novus Biologicals, Littleton, Colo.) and MMP-2 (1:50) (Chemicon Internationals Inc., Temecula, Calif.) overnight at 4° C. in PBS containing 1% bovine serum albumin as a blocking agent. The samples were washed and treated with peroxidase-labeled secondary antibodies at a dilution of 1:100 for 2 h at room temperature. Color development was achieved using diaminobenzidine-H₂O₂. Tissues were counter stained with Alcian blue, mounted in Permount and viewed by light microscopy.

Nucleus pulposus cells reacted strongly with the HIF-1β antibody, although the staining was diffuse. A low level of staining was observed for annulus fibrosus cells, and of those cells that were stained, the majority were from the inner one-third of the tissue. Only the hypertrophic chondrocytes of the end plate tissues were HIF-1 β positive.

The presence of HIF-1α staining in disc cells was also examined and, even when samples were treated by antigen presentation procedures, the cells displayed very low levels of stain in all regions of the intervertebral disc. Similarly, MMP-2 staining was limited to nucleus pulposus cells and very little staining was detected in either the annulus or the end plate cartilage.

RT-PCR Analysis of Disc Cells

The phenotype of nucleus pulposus cells is defined using RT-PCR techniques, which are used in a semi-quantitative manner. If required, Northern analysis is used to study of a particular set of genes at a specified time interval. Total RNA is extracted from the cells with Trizol reagent (Gibco BRL) following the manufacturer's protocol. RT-PCR is performed using Gene Amp kit (Perkin Elmer Corp).

The number of cycles is adjusted so that the reaction is performed within the linear range. Denaturing agarose gel electrophoresis is used to assess the amount and integrity of the RNA. Aggrecan is assayed using the Western Blot analysis.

### Example 7

### Nucleus Pulposus Cell Culture in the Rotating Wall Vessel System

Nucleus pulposus and annulus fibrosus tissues are removed from adult rats approximately 8 to 10 weeks of age. The discs are immersed in HBSS supplemented with 80 mM NaCl. Cells of the nucleus puposus and cells of the inner region of the annulus fibrosus are treated with collagenase at 1 unit/ml for 15 min and 2 hr. respectively, at 37° C. Following collagenase treatment, the cells are swollen and easily ruptured and are gently pipetted up and down to break up the aggregates. The cell suspensions are centrifuged at 2500 rpm for 5 min and the supernatant is discarded and the cell pellet is suspended in complete Dulbecco's Eagle's Medium supplemented with 10% fetal calf serum, 2 mM glutamine and penicillin/streptomycin/fungicide. The cells are treated with hylauronidase (1 unit/ml) to facilitate cell attachment and plated in 60 mm dishes, and the medium is changed at select intervals. To monitor cell growth, the cells are counted in a hemocytometer and the DNA concentration is measured.

Since the microcarriers are of low density and therefore float in regular monolayer culture, the cells (1×10⁶/ml) are injected into the RWVs with the microcarriers at a ratio of cells to microcarriers of about 100:1. The RWV are rotated at a speed of 14 rpm. The oxygen concentration of the medium is regulated and varied from 0.2% to 20%. The ionic strength of the medium is adjusted using NaCl to between 280 and 450 mOsmols. The pH is adjusted by the addition of 10 mM HEPES. The medium is replenished at intervals. Controls include cells maintained on microcarriers in static RWV and cells on microcarriers cultured in plastic culture dishes. Development of the culture is monitored by removing aliquots of microcarriers every two days and determining the DNA content of the cells, which is an indicator of cell growth.

### Example 8

### Evacuation of the Nucleus Pulposus

Mature New Zealand rabbits weighing 4-5 kg are used. For each rabbit, L4-L5 or, when possible L4-L5 and L5-L6 disc spaces are accessed as those are the biggest sections. The anesthetics Ketamine, HCl 30 mg/kg, and Xylazine 6 mg/kg, are administered intramuscularly. Using a paraspinal posterolateral splitting approach, the large cephalad-facing transverse process of the lumbar spine is identified and removed with a rongeur. The intervertebral disc can then be seen. An incision is made in the annulus fibrosus. Using a high-power surgical microscope, the nucleus pulposus tissue is scraped out carefully with a curette. The space is then packed with gel foam. The rabbit is closed provisionally.

### Example 9

### Isolation of Intervertebral Disc Cells

Intervertebral disc tissue is obtained as described in Example 7 or from an amputated tail section. Under aseptic conditions, the intervertebral disc tissue is diced with a scalpel and placed in a T25 tissue culture flask with Dulbecco's Modified Eagle Medium (DMEM) adjusted to pH 7.0, supplemented with 10% heat inactivated fetal bovine serum and 1% penicillin/streptomycin (TCM). The tissue is then treated with 0.25% collagenase for two hours at 37° C. An equal amount of TCM to collagenase is added to stop treatment. The mixture is centrifuged at 1000 r/min for 10 minutes and supernatant is discarded. TCM is added and the mixture is filtered to remove debris. The mixture is again centrifuged and supernatant discarded. Cells are resuspended in TCM supplemented with 1% hyaluronidase (400 u/ml).

### Example 10

### Implantation of Nucleus Pulposus Cells

The rabbit treated as described in Example 7 is reopened per the surgical technique described in Example 7, and the intervertebral disc space accessed. The gel foam is retrieved and nucleus pulposus cell-microcarrier material is inserted. The wound is closed.

What is claimed:

1. A preparation of nucleus pulposus cells, at least 80% by number of the cells of which preparation are nucleus pulposus cells and which nucleus pulposus cells are present in a number effective for accomplishing the reformation of intervertebral disc tissue.

2. The preparation of claim 1, between about 85% and 95% by number of the cells of which are nucleus pulposus cells.

3. The preparation of claim 1, wherein at least about 96% by number of the cells of which are nucleus pulposus cells.

4. The preparation of claim 1 further comprising buffered salt solution.

5. The preparation of claim 1 further comprising extracellular matrix.

6. The preparation of claim 1, wherein the nucleus pulposus cells are generated by isolating nucleus pulposus cells from an intervertebral disc.

7. The preparation of claim 1, wherein the nucleus pulposus cells are generated by culturing nucleus pulposus cells under conditions effective to maintain the phenotype of the nucleus pulposus cells.

8. The preparation of claim 7, wherein the nucleus pulposus cells are combined with a carrier prior to, simultaneous with, or following culturing.

9. The preparation of claim 8, wherein the carrier is one of bioactive glass, metal fiber mesh, or combination thereof.

10. The preparation of claim 8, wherein the carrier comprises bioactive glass.

11. The preparation of claim 8, wherein the carrier is porous.

12. The preparation of claim 8, wherein the nucleus pulposus cells and carrier are combined with at least one biologically active molecule.

13. The preparation of claim 8, wherein the nucleus pulposus cells are bound to the carrier.

14. The preparation of claim 6, wherein the nucleus pulposus cells have been cultured under hypoxic conditions.

15. The preparation of claim 7, wherein the nucleus pulposus cells are cultured under hypoxic conditions.

16. The preparation of claim 7, wherein maintenance of the phenotype of the nucleus pulposus cells is determined by examination of the morphological characteristics of the nucleus pulposus cells.

17. The preparation of claim 7, wherein maintenance of the phenotype of the nucleus pulposus cells is determined using phenotypic markers.

18. The preparation of claim 17, wherein the phenotypic markers are HIF-1α HIF-1β, MMP-2, MMP-9, GLUT-1, LDH-A or Thrombospondin I.

19. The preparation of claim 1, wherein the nucleus pulposus cells are generated by culturing precursor cells under conditions effective to cause the precursor cells to differentiate into nucleus pulposus cells.

20. The preparation of claim 19, wherein the precursor cells are combined with a carrier prior to, simultaneous with, or following culturing.

21. The preparation of claim 20, wherein the carrier is one of bioactive glass, metal fiber mesh, or combination thereof.

22. The preparation of claim 20, wherein the carrier comprises bioactive glass.

23. The preparation of claim 20, wherein the carrier is porous.

24. The preparation of claim 20, wherein the precursor cells and carrier are combined with at least one biologically active molecule.

25. The preparation of claim 20, wherein the nucleus pulposus cells are bound to the carrier.

26. The preparation of claim 19, wherein the precursor cells are cultured under hypoxic conditions.

27. The preparation of claim 19, wherein differentiation of the precursor cells into nucleus pulposus cells is determined by examination of the morphological characteristics of the nucleus pulposus cells.

28. The preparation of claim 19, wherein differentiation of the precursor cells into nucleus pulposus cells is determined using phenotypic markers.

29. The preparation of claim 28, wherein the phenotypic markers are HIF-1α, HIF-1β, MMP-2, MMP-9, GLUT-1, LDH-A or Thrombospondin I.

30. A preparation of nucleus pulposus cells comprising nucleus pulposus cells in an amount of at least 80% by number that are generated by culturing nucleus pulposus cells under conditions effective to maintain the phenotype of the nucleus pulposus cells.

31. The preparation of claim 30, wherein the nucleus pulposus cells are combined with a carrier prior to, simultaneous with, or following culturing.

32. The preparation of claim 31, wherein the carrier is one of bioactive glass, metal fiber mesh, or combination thereof.

33. The preparation of claim 31, wherein the carrier comprises bioactive glass.

34. The preparation of claim 31, wherein the carrier is porous.

35. The preparation of claim 31, wherein the nucleus pulposus cells and carrier are combined with at least one biologically active molecule.

36. The preparation of claim 31, wherein the nucleus pulposus cells are bound to the carrier.

37. The preparation of claim 30, wherein the nucleus pulposus cells are cultured under hypoxic conditions.

38. The preparation of claim 30, wherein maintenance of the phenotype of the nucleus pulposus cells is determined by examination of the morphological characteristics of the nucleus pulposus cells.

39. The preparation of claim 30, wherein maintenance of the phenotype of the nucleus pulposus cells is determined using phenotypic markers.

40. The preparation of claim 39, wherein the phenotypic markers are HIF-1α, HIF-1β, MMP-2, MMP-9, GLUT-1, LDH-A or Thrombospondin I.

41. A preparation of nucleus pulposus cells comprising nucleus pulposus cells in an amount of at least 80% by number generated in vitro from precursor cells by culturing the precursor cells under conditions effective to cause the precursor cells to differentiate into said nucleus pulposus cells.

42. The preparation of claim 41, wherein the precursor cells are combined with a carrier prior to, simultaneous with, or following culturing.

43. The preparation of claim 42, wherein the carrier is one of bioactive glass, metal fiber mesh, or combination thereof.

44. The preparation of claim 42, wherein the carrier comprises bioactive glass.

45. The preparation of claim 42, wherein the carrier is porous.

46. The preparation of claim 42, wherein the precursor cells and carrier are combined with at least one biologically active molecule.

47. The preparation of claim 42, wherein the nucleus pulposus cells are bound to the carrier.

48. The preparation of claim 41, wherein the precursor cells are cultured under hypoxic conditions.

49. The preparation of claim 41, wherein differentiation of the precursor cells into nucleus pulposus cells is determined by examination of the morphological characteristics of the nucleus pulposus cells.

50. The preparation of claim 41, wherein differentiation of the precursor cells into nucleus pulposus cells is determined using phenotypic markers.

51. The preparation of claim 50, wherein the phenotypic markers are HIF-1α, HIF-1β, MMP-2, MMP-9, GLUT-1, LDH-A or Thrombospondin I.

52. A method of treating degenerative intervertebral disc disease in an individual comprising implanting nucleus pulposus cells into the nucleus pulposus space of a degenerated disc of the individual.

53. The method of claim 52, wherein the nucleus pulposus cells are generated by isolating nucleus pulposus cells from an intervertebral disc.

54. The method of claim 52, wherein the nucleus pulposus cells are generated by culturing nucleus pulposus cells under conditions effective to maintain the phenotype of the nucleus pulposus cells.

55. The method of claim 54, further comprising combining the nucleus pulposus cells with a carrier prior to, simultaneous with, or following culturing.

56. The method of claim 55, wherein the carrier comprises bioactive glass.

57. The method of claim 55, further comprising combining the nucleus pulposus cells and carrier with at least one biologically active molecule.

58. The method of claim 53, wherein the nucleus pulposus cells have been cultured under hypoxic conditions.

59. The method of claim 54, wherein the nucleus pulposus cells are cultured under hypoxic conditions.

60. The method of claim 54, wherein maintenance of the phenotype of the nucleus pulposus cells is determined by examination of the morphological characteristics of the nucleus pulposus cells.

61. The method of claim 54, wherein maintenance of the phenotype of the nucleus pulposus cells is determined using phenotypic markers.

62. The method of claim 61, wherein the phenotypic markers are HIF-1α, HIF-1β, MMP-2, MMP-9, GLUT-1, LDH-A or Thrombospondin I.

63. The method of claim 52, wherein the nucleus pulposus cells are generated by culturing precursor cells under conditions effective to cause the precursor cells to differentiate into nucleus pulposus cells.

64. The method of claim 63, wherein the precursor cells comprise at least one of annulus fibrosus and nucleus pulposus cells.

65. The method of claim 63, wherein the precursor cells are isolated from an intervertebral disc.

66. The method of claim 65, wherein the precursor cells are isolated from an intervertebral disc of the individual to be treated.

67. The method of claim 65, wherein the precursor cells are treated with hylauronidase prior to culturing.

68. The method of claim 63, wherein the precursor cells are combined with a carrier prior to, simultaneous with, or following culturing.

69. The method of claim 68, further comprising culturing the precursor cells prior to combining the precursor cells with the carrier.

70. The method of claim 68, further comprising combining the precursor cells and carrier with at least one biologically active molecule.

71. The method of claim 70, wherein the biologically active molecule is a growth factor, cytokine, antibiotic, protein, anti-inflammatory agent, or analgesic.

72. The method of claim 70, wherein the biologically active molecules are contained within or upon the carrier.

73. The method of claim 70, wherein the biologically active molecules are released from the carrier in a controlled release manner.

74. The method of claim 68, wherein the carrier comprises bioactive glass.

75. The method of claim 74 wherein the bioactive glass comprises 45S5 glass.

76. The method of claim 63, wherein the precursor cells are cultured under hypoxic conditions.

77. The method of claim 76, wherein the precursor cells are cultured in a medium in which the oxygen concentration is maintained at from about 0.2% to about 2%.

78. The method of claim 63, wherein the precursor cells are cultured in a medium in which the ionic strength is maintained at from about 100 mOsmols to about 900 mOsmols.

79. The method of claim 78, wherein the precursor cells are cultured in a medium in which the ionic strength is maintained at from about 280 mOsmols to about 450 mOsmols.

80. The method of claim 63, wherein the precursor cells are cultured in a medium comprising fibronectin.

81. The method of claim 63, wherein differentiation of the precursor cells into nucleus pulposus cells is determined by examination of the morphological characteristics of the nucleus pulposus cells.

82. The method of claim 63, wherein differentiation of the precursor cells into nucleus pulposus cells is determined using phenotypic markers.

83. The method of claim 82, wherein the phenotypic markers are indicative of hypoxic conditions.

84. The method of claim 82, wherein the phenotypic markers are HIF-1α, HIF-1β, MMP-2, MMP-9, GLUT-1, LDH-A or Thrombospondin I.

85. A method of generating nucleus pulposus cells comprising culturing nucleus pulposus cells under conditions effective to maintain the phenotype of the nucleus pulposus cells.

86. The method of claim 85 using a rotating wall vessel.

87. The method of claim 85, further comprising combining the nucleus pulposus cells with a carrier prior to, simultaneous with, or following culturing.

88. The method of claim 87, wherein the carrier comprises bioactive glass.

89. The method of claim 87, further comprising combining the nucleus pulposus cells and carrier with at least one biologically active molecule.

90. The method of claim 85, wherein the nucleus pulposus cells are cultured under hypoxic conditions.

91. The method of claim 85, wherein maintenance of the phenotype of the nucleus pulposus cells is determined by examination of the morphological characteristics of the nucleus pulposus cells.

92. The method of claim 85, wherein maintenance of the phenotype of the nucleus pulposus cells is determined using phenotypic markers.

93. The method of claim 92, wherein the phenotypic markers are HIF-1α, HIF-1β, MMP-2, MMP-9, GLUT-1, LDH-A or Thrombospondin I.

94. A three-dimensional matrix produced by the method of claim 87.

95. A method of generating nucleus pulposus cells comprising culturing precursor cells under conditions effective to cause the precursor cells to differentiate into nucleus pulposus cells.

96. The method of claim 95 using a rotating wall vessel.

97. The method of claim 95, further comprising combining the precursor cells with a carrier prior to, simultaneous with, or following culturing.

98. The method of claim 97, wherein the carrier comprises bioactive glass.

99. The method of claim 97, further comprising combining the precursor cells and carrier with at least one biologically active molecule.

100. The method of claim 95, wherein the precursor cells are cultured under hypoxic conditions.

101. The method of claim 95, wherein differentiation of the precursor cells into nucleus pulposus cells is determined by examination of the morphological characteristics of the nucleus pulposus cells.

102. The method of claim 95, wherein differentiation of the precursor cells into nucleus pulposus cells is determined using phenotypic markers.

103. The method of claim 102, wherein the phenotypic markers are HIF-1α, HIF-1β, MMP-2, MMP-9, GLUT-1, LDH-A or Thrombospondin I.

104. A three-dimensional matrix produced by the method of claim 97.

105. A method of treating degenerative intervertebral disc disease in an individual comprising the steps of:
(a) isolating precursor cells from a sample;
(b) seeding the cells onto a carrier;
(c) culturing the cells under conditions effective to cause the precursor cells to differentiate into nucleus pulposus cells; and
(d) implanting the nucleus pulposus cells into the nucleus pulposus space of a degenerated disc of the individual.

106. The method of claim 105, wherein the sample comprises an intervertebral disc.

107. The method of claim 106, wherein the intervertebral disc is obtained from the individual.

108. The method of claim 105, wherein the sample comprises stem cells.

109. The method of claim 105, wherein the precursor cells comprise annulus fibrosus cells.

110. A method of identifying nucleus pulposus cells comprising the steps of:
(a) obtaining a sample; and
(b) detecting evidence of expression of nucleus pulposus phenotypic markers selected from the group consisting of HIF-1α, HIF-1β, MMP-2, MMP-9, GLUT-1, LDH-A and Thrombospondin I in said sample,
wherein evidence of expression of HIF-1α, HIF-1β, MMP-2, MMP-9, GLUT-1, LDH-A or Thrombospondin I in said sample indicates the presence of nucleus pulposus cells in said sample.

111. The method of claim 110 wherein the sample is obtained from an intervertebral disc of an individual.
